(19) United States
(12) Patent Application Publication
(10) Pub. No.: US 2010/0233747 A1
(43) Pub. Date: Sep. 16, 2010

(54) METHOD AND MEANS FOR THE ENZYMATIC DETERMINATION OF ETHANOL

(76) Inventor: Ralf Olt, Luetzelbach-Seckmauern (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/301,778

(22) PCT Filed: Apr. 23, 2007
(86) PCT No.: PCT/EP07/03534

§ 371 Date: Nov. 21, 2008

(30) Foreign Application Priority Data
May 22, 2006 (DE) 10 2006 023 897.4

(51) Int. Cl. C12Q 1/32

(52) U.S. Cl. 435/26

(57) ABSTRACT

The invention relates to a method and means for the enzymatic determination of ethanol. The method according to the invention prevents ethanol vapours in the ambient air from interfering with the determination.

The invention relates to a method and means for the enzymatic determination of ethanol. The method according to the invention prevents ethanol vapours in the ambient air from interfering with the determination.

The determination of ethanol plays an important role in many areas, such as, for example, in industry for the monitoring of fermentation processes or for determination of the purity of reagents, in the foods industry for the determination of the alcohol content of foods and drinks, or in medicine for the determination of the blood alcohol level.

The alcohol content of samples can differ greatly. Whereas small amounts of alcohol have to be detected in the determination of the blood alcohol content, it is necessary to be able to determine a high ethanol content accurately in, for example, alcoholic beverages.

Many determination methods are known in the prior art. In some cases, considerable technical complexity is necessary for carrying out these methods. On the other hand, there are enzymatic determination methods, which can be carried out quickly and simply. These methods can be carried out wet-chemically or dry-chemically, i.e. with the aid of a test stick. An over-view of the most important methods for the determination of ethanol is given in EP 0 164 008 on page 2.

EP 0 164 008 furthermore relates to a dry-chemical enzymatic determination of ethanol in which the ethanol present in a sample is reacted with an alcohol oxidase in the presence of oxygen. The hydrogen peroxide forming then causes a visible coloration with peroxidase and a chromogenic substrate, the intensity of which can be measured for the determination of the alcohol concentration.

A further method for the enzymatic determination of ethanol is disclosed in H.-O. Beutler (Ethanol, in H. U. Bergmeyer (ed.), Methods of Enzymatic Analysis, 3rd Edition, Weinheim 1984, Vol. 6, pages 598-606). Here, ethanol is converted into acetaldehyde in the presence of alcohol dehydrogenase (ADH) and NAD. Acetaldehyde is in turn converted into acetate by means of NAD and aldehyde dehydrogenase. In both reaction steps, NADH is formed. The NADH formed can be converted into a coloured formazane in a known manner with reduction of a tetrazolium salt.

DD 256 196 A1 and U.S. Pat. No. 5,294,540 also disclose dry-chemical methods for the determination of the ethanol content in body fluids, in which ethanol is converted into acetaldehyde in the presence of alcohol dehydrogenase (ADH) and NAD.

These methods have the disadvantage, especially if they are carried out on test sticks, that alcohol present in the ambient air reacts with the indicator system and falsifies the measurement results. This interference plays an important role, in particular, in industry in the monitoring of fermentation processes, in the determination of the purity of reagents and in the foods industry for the determination of the alcohol content of foods and drinks. Reliable results are obtained with alcohol test strips only if particular precautions are taken to exclude these adverse effects from the ambient air.

The object of the present invention was to develop a method by means of which an ethanol determination can be carried out in such a way that interference by ethanol vapours from the ambient air can be minimised or completely excluded.

It has been found that it is possible to eliminate the interference of the determination by ethanol vapours from the ambient air if the reaction is carried out in two steps, where, in the first step, the enzymatic conversion of the ethanol is carried out in the presence of alcohol dehydrogenase (ADH) and NAD, and, in a second step, the reflectometric or colorimetric determination of the reaction product NADH formed is carried out, where the enzymatic activity of the alcohol dehydrogenase in the second step is simultaneously suppressed.

The present invention therefore relates to a method for the determination of the ethanol content of a sample, characterised by the following method steps:
a) provision of a sample whose ethanol content is to be determined
b) bringing the sample into contact with a reagent composition A which comprises at least alcohol dehydrogenase, NAD(P) and a buffer which produces a pH of greater than or equal to pH 7.5, and either a compound which is able to form a Schiff base with acetaldehyde, or aldehyde dehydrogenase (AIDH)
c) incubation of the mixture from step b)
d) bringing the mixture from step c) into contact with a reagent composition B which comprises at least reagents for the colorimetric determination of NAD(P)H and inhibits alcohol dehydrogenase
e) incubation of the mixture from step d)
f) determination of the ethanol content from the colour development forming

In a preferred embodiment, the reagent composition A from step b) is a reagent tablet.

In a preferred embodiment, the reagent composition B from step d) is a test stick.

In a preferred embodiment, the reagent composition A from step b) comprises NAD.

In a preferred embodiment, the reagent composition B from step d) comprises 4-iodopyrazole as inhibitor of alcohol dehydrogenase.

In a preferred embodiment, the reagent composition B from step d) comprises at least nitro blue tetrazolium chloride and diaphorase as reagents for the colorimetric determination of NAD(P)H.

In another preferred embodiment, the reagent composition B from step d) comprises at least nitro blue tetrazolium chloride and 1-methoxy-5-methylphenazinium methylsulfate as reagents for the colorimetric determination of NAD(P)H, and a buffer which produces a pH of less than or equal to 6.

In a preferred embodiment, the determination of the ethanol content in step f) is carried out by reflectometry.

The present invention also relates to a test set for the determination of the ethanol content of a sample, at least comprising
a) a reagent composition A which comprises at least alcohol dehydrogenase, NAD(P) and a buffer which produces a pH of greater than or equal to pH 7.5, and either a compound which is able to form a Schiff base with acetaldehyde, or aldehyde dehydrogenase (AIDH) and
b) a reagent composition B which comprises at least reagents for the colorimetric determination of NAD(P)H and inhibits alcohol dehydrogenase.

In a preferred embodiment, both reagent compositions comprise, independently of one another, buffers and stabilisers.

In a preferred embodiment, the reagent composition A is in the form of a reagent tablet, and the reagent composition B is in the form of a test stick.

In the present specification, the following abbreviations are used:

NAD nicotinamide adenine dinucleotide (oxidised form)

NAD(P) nicotinamide adenine dinucleotide and/or nicotinamide adenine dinucleotide phosphate (oxidised form)

NAD(P)H nicotinamide adenine dinucleotide and/or nicotinamide adenine dinucleotide phosphate (reduced form)

NADH nicotinamide adenine dinucleotide (reduced form)

NADP nicotinamide adenine dinucleotide phosphate (oxidised form)

NADPH nicotinamide adenine dinucleotide phosphate (reduced form)

NBT nitro blue tetrazolium chloride

In accordance with the invention, a sample is typically liquid. The sample can be, for example, a solution or suspension, which generally comprises water or a buffer solution as predominant solvent. Solid samples are firstly dissolved or suspended in water or a buffer solution or extracted therewith. Examples of samples are dilute juices, alcoholic beverages or blood. Depending on the nature of the sample and the alcohol content thereof, it may be advantageous to dilute the sample with water or a buffer solution before carrying out the determination according to the invention.

In accordance with the invention, a colorimetric determination is a determination in which a product which is visibly coloured in the visual region and can be evaluated visually, preferably semi-quantitatively or quantitatively, is formed. The evaluation is preferably carried out by reflectometry, for example using simple reflectometers.

Analysis using solid, sorptive supports, so-called test sticks, has recently increased in importance. The main advantages of these dry-chemical methods include, in particular, simple handling and straightforward disposal owing to the small amounts of reagent. All or the majority of the reagents necessary for the determination reaction are embedded in corresponding layers of a solid, sorptive or swellable support to which the sample is applied. After contact of the reaction zone with the sample, the determination reaction proceeds. The colour formed is a measure of the amount of analyte to be determined and can be evaluated visually, i.e. semi-quantitatively, or quantitatively using simple reflectometers.

Sorptive supports which can be used for the test strips of the test set according to the invention are all materials which are usually used for dry-chemical tests of this type. The most widespread is the use of filter paper, but other sorptive cellulose or plastic products can also be employed.

The sorptive supports are impregnated with impregnation solutions in a known manner. The impregnation solutions preferably comprise all reagents necessary for the determination of the analyte.

The impregnated and dried supports can be cut to a suitable size and adhesively bonded or heat-sealed to support films or foils in a known manner for the production of test strips.

The sorptive support to which the determination reagents are applied usually does not cover the entire test strip, but instead merely a zone of the test strip. In this way, it is possible to combine not only one zone having one composition of determination reagents, but instead a plurality of zones having identical or different compositions on a single test strip. In accordance with the invention, the region of the test strip to which the reagents necessary for the determination of an analyte or for the recording of a blank value are applied on a sorptive support is therefore referred to as a zone.

The ethanol content determination according to the invention is carried out in two steps. Firstly, the ethanol in the sample is reacted with alcohol dehydrogenase and NAD(P) (method steps a) to c) of the method according to the invention). In the process, the ethanol is converted into acetaldehyde, while NAD(P) is reduced to NAD(P)H. In accordance with the invention, this reaction step is carried out in solution. To this end, the sample is mixed or brought into contact with a reagent composition A which comprises at least alcohol dehydrogenase, NAD(P) and a buffer which produces a pH of greater than or equal to pH 7.5, and either a compound which is able to form a Schiff base with acetaldehyde, or aldehyde dehydrogenase (AIDH).

The presence of aldehyde dehydrogenase or the compound which is able to form a Schiff base with acetaldehyde removes acetaldehyde from the reaction mixture and thus shifts the equilibrium of the reaction in the direction of acetaldehyde formation. Further details on the use of aldehyde dehydrogenase in this reaction of ethanol are given in H.-O-Beutler (Ethanol, in H. U. Bergmeyer (ed.), Methods of Enzymatic Analysis, 3rd Edition, Weinheim 1984, Vol. 6, pages 598-606, in particular on pages 601-603).

The reagent composition A preferably comprises at least alcohol dehydrogenase and NAD, and a compound which is able to form a Schiff base with acetaldehyde and a buffer which produces a pH of greater than or equal to pH 7.5.

Buffer substances which are able to produce a pH greater than or equal to pH 7.5 are known to the person skilled in the art. Preference is given in accordance with the invention to the use of TRIS buffer (tris(hydroxymethyl)-aminomethane, since TRIS is able to produce a suitable basic pH and at the same time represents a compound which is able to form a Schiff base with acetaldehyde.

The reagent composition A preferably comprises buffer substances which produce a pH between 8 and 10.

Compounds which are able to form a Schiff base with acetaldehyde are, in particular, amines, hydrazines, semicarbazides or hydroxylamines. Preference is given to the use of hydrazine or hydroxylamine, particularly preferably TRIS.

Since TRIS, as explained above, is both suitable for producing a pH greater than or equal to pH 7.5 and is also able to form a Schiff base with acetaldehyde, TRIS is particularly preferably employed. In this case, one compound, namely IRIS, meets both requirements.

Alcohol dehydrogenase and NAD(P) are present in sufficient amount that rapid and complete conversion of the ethanol is ensured.

The reagent composition A can be in liquid form as a solution or in solid form, for example as a powder, capsule or reagent tablet. The reagent composition is preferably in the form of a tablet. The reagent composition A typically comprises further constituents, such as buffer substances, stabilisers or optionally tabletting assistants.

If reagent composition A is in the form of a reagent tablet, it can, for example, have the following composition:

| | | |
|---|---|---|
| 25-30 | mg | of tris(hydroxymethyl)aminomethane |
| 1-1.8 | mg | of glycine |
| 10-15 | U | of alcohol dehydrogenase |
| 5-6 | mg | of nicotinamide adenine dinucleotide |
| | | fillers and stabilisers |

The reagent tablet preferably has the following composition:

| | | |
|---|---|---|
| 28.4 | mg | of tris(hydroxymethyl)aminomethane |
| 1.42 | mg | of glycine |
| 13.4 | U | of alcohol dehydrogenase |
| 5.58 | mg | of nicotinamide adenine dinucleotide |
| to 67.5 | mg | of fillers and stabilisers |

In order to ensure the most complete conversion possible of the ethanol in the sample, the mixture of sample and reagent composition A is incubated. The incubation time is typically between 30 seconds and 10 minutes. The incubation is preferably carried out at temperatures between 18 and 37° C., particularly preferably at room temperature.

H.-O-Beutler (Ethanol, in H. U. Bergmeyer (ed.), Methods of Enzymatic Analysis, 3rd Edition, Weinheim 1984, Vol. 6, pages 598-606) describes in detail how the determination of ethanol using alcohol dehydrogenase and NAD(P) can be carried out.

In accordance with the invention, the concentration of the NAD(P)H formed is subsequently determined in a second step (method steps d) to f) of the method according to the invention). This is carried out by mixing or bringing the mixture resulting from method step c) with or into contact with a reagent composition B which comprises at least reagents for the colorimetric determination of NAD(P)H and inhibits alcohol dehydrogenase. The inhibition of the alcohol dehydrogenase can be carried out by addition of an inhibitor to reagent composition B or by reagent composition B producing a pH which deactivates alcohol dehydrogenase.

The reagent composition B can be in liquid form as a solution, in solid form, for example as a powder, capsule or reagent tablet, or as a test stick. The reagent composition is preferably in the form of a test stick. All reagents necessary for the determination of NAD(P)H are preferably present on the test stick.

The reagent composition B typically comprises reagents for the colorimetric determination of NAD(P)H, an inhibitor of alcohol dehydrogenase or buffer substances for producing a pH which deactivates the alcohol dehydrogenase, and optional further constituents, such as buffer substances, stabilisers or tabletting assistants.

If the colorimetric determination of NAD(P)H is carried out enzymatically by means of diaphorase with reduction of a tetrazolium salt to a coloured formazane, this reaction should be carried out at a pH between 7 and 9 in order to ensure adequate activity of the diaphorase. At pH values between 7 and 9, however, the alcohol dehydrogenase is also active, and consequently an inhibitor of alcohol dehydrogenase is in this case added in accordance with the invention to the reagent composition b).

Alcohol dehydrogenase inhibitors which can be used are all known alcohol dehydrogenase inhibitors which do not interfere with the determination of NAD(P)H. These are, for example, pyrazole, 4-alkylpyrazoles and 4-halogenated pyrazoles, preferably 4-iodopyrazole.

Both the enzyme diaphorase and also the tetrazolium salt and the inhibitor of alcohol dehydrogenase are employed in concentrations such that rapid reaction takes place. A rapid reaction preferably means reaction times of less than 15 minutes.

In a preferred embodiment, the conversion of the NAD(P)H formed in method step b) or c) into a coloured formazane is not carried out enzymatically, but instead by means of a substance such as phenazine methosulfate or phenazine ethosulfate or derivatives thereof, such as, for example, preferably 1-methoxy-5-methylphenazinium methylsulfate (MPMS) with reduction of a tetrazolium salt. In this case, the inhibition of the alcohol dehydrogenase can be carried out by means of an inhibitor or by adjusting the pH into a range in which alcohol dehydrogenase is no longer active. The inhibition of alcohol dehydrogenase is preferably effected by changing the pH. The inhibition of alcohol dehydrogenase is particularly effective on establishment of a pH below pH 6, preferably between 2 and 5.5.

In a particularly preferred embodiment, reagent composition b) therefore comprises at least phenazine methosulfate, phenazine ethosulfate or a derivative thereof (preferably 1-methoxy-5-methylphenazinium methylsulfate (MPMS)) and a tetrazolium salt, meaning that the NAD(P)H formed is converted into a coloured formazane by phenazine methosulfate or a derivative thereof with reduction of the tetrazolium salt. The reagent composition B furthermore comprises a buffer which establishes a pH less than or equal to pH 6, preferably between pH 2.0 and pH 5.5, particularly preferably about pH 3.5. In the case of a test stick, this should comprise a buffer which establishes the above-mentioned pH values for the determination reaction, at least in the determination zone.

Suitable buffers are all buffer substances which develop a buffering action in the pH range below about pH 6, preferably between pH 2.0 and 5.5, for example citric acid and salts thereof, malic acid and salts thereof, succinic acid and salts thereof, tartaric acid and salts thereof, diglycolic acid and salts thereof, preferably citric acid/citrate. The concentration of the buffer in the impregnation solution is selected so that the sample taken up by the paper having a pH of, for example, 9.5 is re-buffered by the buffer present in the paper to a pH of, for example, <5.0, for example 425 mmol/l of citric acid/citrate at pH 3.5.

Suitable tetrazolium salts are, for example, NBT or MTT (3-(4,5-dimethylthiazol-2-yl)-2,5-diphenyl-2-tetrazolium bromide).

In order to ensure the most complete conversion possible of the NAD(P)H, the mixture is incubated after bringing into contact with reagent composition B. The incubation time is typically between 30 seconds and 10 minutes. The incubation is preferably carried out at temperatures between 18 and 37° C., particularly preferably at room temperature.

After the end of the reaction, the intensity of the colour of the formazane is determined. This can be carried out visually, for example semi-quantitatively by comparison with a colour chart, or quantitatively, for example by means of a photometer or reflectometer. On use of a test strip, the remission is preferably measured by reflectometry. The concentration of the ethanol in the sample can be calculated therefrom via a calibration curve.

The present invention also relates to a test set for carrying out the method according to the invention.

The test set comprises at least
a) a reagent composition A which comprises at least alcohol dehydrogenase, NAD(P) and a buffer which produces a pH of greater than or equal to pH 7.5, and either a compound which is able to form a Schiff base with acetaldehyde, or aldehyde dehydrogenase (AIDH) and
b) a reagent composition B which comprises at least reagents for the colorimetric determination of NAD(P)H and inhibits alcohol dehydrogenase.

Regarding the composition of the reagent compositions and the preferred embodiments, reference is made to the comments in the description of the method according to the invention.

In a preferred embodiment, the reagent composition A comprises a buffer which produces a pH between pH 8 and 10.

In a particularly preferred embodiment, the buffer is an amine buffer, such as, for example, TRIS.

In a preferred embodiment, reagent composition A is in the form of a reagent tablet and reagent composition B is in the form of a test stick.

It has been found that, on use of the method and test set according to the invention, the interfering influence of ethanol from the ambient air can be avoided. “Be avoided” here means in accordance with the invention that no significant difference in the measurement result occurs in comparative measurements with and without ethanol in the ambient air.

The method according to the invention and the test set according to the invention thus offer a simple, rapid and sensitive method for the enzymatic determination of ethanol. The preferred method procedure with the aid of a test strip enables the determination to be carried out, even by untrained personnel, without complex equipment and without handling toxic chemicals.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below, in particular the corresponding application DE 10 2006 023 897.4, filed on May 22, 2006, is incorporated into this application by way of reference.

### EXAMPLES

### Example 1

Reagent tablets for the determination of ethanol having the following composition are produced:

| | | |
|---|---|---|
| 28.4 | mg | of tris(hydroxymethyl)aminomethane |
| 1.42 | mg | of glycine |
| 13.4 | U | of alcohol dehydrogenase |
| 5.58 | mg | of nicotinamide adenine dinucleotide |
| to 67.5 | mg | fillers and stabilisers |

Test strips having two paper zones are produced, with a solution of the following composition being applied to both zones:

| | | |
|---|---|---|
| 50 | mmol/l | of triethanolamine buffer pH 7.2 |
| 1 | g/l | of nitro blue tetrazolium chloride |
| 85 | U/l | of diaphorase |
| 1 | mol/l | of 4-iodopyrazole |

After drying, these test strips are stored under dry conditions at 2° C. to 8° C.

The determination is carried out as follows:

A reagent tablet is initially introduced into a sealable reagent vessel and dissolved in 0.5 ml of pre-diluted sample. The solution is incubated at room temperature for 4 minutes. The solution is subsequently diluted with 10 ml of water, and the test strip is dipped in. Excess liquid is removed from the test strip, and the test strip is incubated at room temperature for 3 minutes. The formazane colour formed is measured by reflectometry, and the ethanol concentration is calculated via a calibration curve.

### Example 2

Reagent tablets for the determination of ethanol having the following composition are produced:

| | | |
|---|---|---|
| 28.4 | mg | of tris(hydroxymethyl)aminomethane |
| 1.42 | mg | of glycine |
| 13.4 | U | of alcohol dehydrogenase |
| 5.58 | mg | of nicotinamide adenine dinucleotide |
| to 67.5 | mg | fillers and stabilisers |

Test strips having two paper zones are produced, with a solution of the following composition being applied to both zones:

| | | |
|---|---|---|
| 425 | mmol/l | of citrate buffer pH 3.5 |
| 1 | g/l | of nitro blue tetrazolium chloride |
| 6 | μmol/l | of 1-methoxy-5-methylphenazinium methylsulfate |

After drying, these test strips are stored under dry conditions at 2° C. to 8° C.

The determination is carried out as follows:

A reagent tablet is initially introduced into a sealable reagent vessel and dissolved in 0.5 ml of pre-diluted sample. The solution is incubated at room temperature for 4 minutes. The solution is subsequently diluted with 10 ml of water, and the test strip is dipped in. Excess liquid is removed from the test strip, and the test strip is incubated at room temperature for 3 minutes. The formazane colour formed is measured by reflectometry, and the ethanol concentration is calculated via a calibration curve.

1. Method for the determination of the ethanol content of a sample, characterised by the following method steps:
a) provision of a sample whose ethanol content is to be determined
b) bringing the sample into contact with a reagent composition A comprising at least alcohol dehydrogenase, NAD(P) and a buffer which produces a pH of greater than or equal to pH 7.5, and either a compound which is able to form a Schiff base with acetaldehyde, or aldehyde dehydrogenase (AIDH)
c) incubation of the mixture from step b)
d) bringing the mixture from step c) into contact with a reagent composition B which comprises at least reagents for the colorimetric determination of NAD(P)H and inhibits alcohol dehydrogenase
e) incubation of the mixture from step d)
f) determination of the ethanol content from the colour development forming

2. Method according to claim 1, characterised in that the reagent composition A from step b) is a reagent tablet.

3. Method according to claim 1, characterised in that the reagent composition B from step d) is a test stick.

4. Method according to claim 1, characterised in that the reagent composition A from step b) comprises NAD.

5. Method according to claim 1, characterised in that the reagent composition B from step d) comprises 4-iodopyrazole as inhibitor of alcohol dehydrogenase.

6. Method according to claim 1, characterised in that the reagent composition B from step d) comprises at least nitro blue tetrazolium chloride and diaphorase as reagents for the colorimetric determination of NAD(P)H.

7. Method according to claim 1, characterised in that the reagent composition B from step d) comprises at least nitro blue tetrazolium chloride and 1-methoxy-5-methylphenazinium methylsulfate as reagents for the colorimetric determination of NAD(P)H and a buffer which produces a pH of less than or equal to 6.

8. Method according to claim 1, characterised in that the determination of the ethanol content in step 0 is carried out by reflectometry.

9. Test set for the determination of the ethanol content of a sample, at least comprising
a) a reagent composition A which comprises at least alcohol dehydrogenase, NAD(P) and a buffer which produces a pH of greater than or equal to pH 7.5, and either a compound which is able to form a Schiff base with acetaldehyde, or aldehyde dehydrogenase (AIDH) and
b) a reagent composition B which comprises at least reagents for the colorimetric determination of NAD(P)H and inhibits alcohol dehydrogenase.

10. Test set according to claim 9, characterised in that both reagent compositions comprise, independently of one another, buffers and stabilisers.

11. Test set according to claim 9, characterised in that the reagent composition A is in the form of a reagent tablet and the reagent composition B is in the form of a test stick.
